(19) United States
(12) Patent Application Publication
(10) Pub. No.: US 2025/0025588 A1
(43) Pub. Date: Jan. 23, 2025

(54) ENHANCED ULTRAVIOLET SANITIZATION

(71) Applicant: Robert E SANDSTROM, Longview, WA (US)

(72) Inventor: Robert E SANDSTROM, Longview, WA (US)

(21) Appl. No.: 18/775,425

(22) Filed: Jul. 17, 2024

Related U.S. Application Data
(60) Provisional application No. 63/513,922, filed on Jul. 17, 2023.

(51) Int. Cl. A61L 2/10

(52) U.S. Cl. CPC A61L 2/10; A61L 2202/122; A61L 2202/21; A61L 2202/24; A61L 2202/26

(57) ABSTRACT

Methods and apparatus for sanitizing and/or sterilizing a material, including placing a material to be sanitized and/or sterilized into a reaction chamber, and simultaneously exposing the material to be sanitized and/or sterilized to an ultraviolet light selected to decrease a number of living or viable microorganisms in or on the material, and to a magnetic field having a field strength selected to prolong a lifetime of a free radical entity generated by the ultraviolet light exposure for a time sufficient to prolong the lifetime of the free radical entity.

### TECHNICAL FIELD

The present disclosure relates to methods for sanitizing and/or sterilizing objects and surfaces, and more particularly to the use of ultraviolet light to sanitize and/or sterilize objects and surfaces.

### BACKGROUND

Ultraviolet light (UV) corresponds to a region of the electromagnetic spectrum having wavelengths shorter than that of visible light, but longer than that of X-rays. UV radiation is present in sunlight, constituting about 10% of the total electromagnetic radiation output from the Sun, but is also produced by electric arcs, Cherenkov radiation, and specialized lights, such as mercury-vapor lamps, tanning lamps, and black lights.

The use of ultraviolet wavelength of light as a germicide can be traced back to 1878, when Arthur Downes and Thomas Blunt demonstrated that the shorter wavelengths of sunlight could inactivate certain bacteria.

UV light Induces photolytic effects which can create free radicals, or chemical moieties with an unpaired electron in an outer electron orbital. Free radical entities possess an unpaired electron, and are therefore subject to the effects of an applied magnetic field. In particular, free radical entities precess in a magnetic field of a specific strength. The magnetic field strength required for such precession is dictated by the quantum state of the radical within the magnetic field. Precession in the magnetic field is comparable to classical Larmor Precession, while the dynamics of free radical behavior in quantum terms is governed by the Pauli Exclusion Principle by virtue of the electron spin state of the radical.

A specific and relatively low strength magnetic field can therefore prolong free radical reactivity and limit immediate free radical recombination.

### SUMMARY

The present disclosure is directed to methods and apparatus for sanitizing and/or sterilizing a material, including placing a material to be sanitized and/or sterilized into a reaction chamber, and simultaneously exposing the material to be sanitized and/or sterilized to an ultraviolet light selected to decrease a number of living or viable microorganisms in or on the material, and to a magnetic field having a field strength selected to prolong a lifetime of a free radical entity generated by the ultraviolet light exposure for a time sufficient to prolong the lifetime of the free radical entity.

### DETAILED DESCRIPTION

Ultraviolet germicidal irradiation is an increasingly significant method for sanitizing and/or sterilizing surfaces, food, air, and water. Ultraviolet germicidal irradiation is also used to decontaminate and sterilize medical devices and instruments.

As used herein, the terms sanitizing and sterilizing are closely related, and refer to a process for decreasing the number of living or viable microorganisms on or in a material or surface. In one aspect of the disclosure, the term sanitizing describes lowering the number of such living or viable microorganisms on or in a material by 99.9%. In another aspect of the disclosure, particularly with respect to the sanitization of hard surfaces and/or surfaces used to food service, sanitizing describes lowering the number of such living or viable microorganisms on such a surface by 99.99%. In another aspect of the disclosure, the term sterilizing describes lowering the number of such living or viable microorganisms on or in a material by 99.999%.

All current UV-employing germicidal methods, whether employing UVA, UVB, or UVC, produce germicidal effects through the generation of free radicals. In particular, UVC irradiation employs ultraviolet frequencies between 255 and 280 nanometers. The introduction of reliable UVC light-emitting diodes has allowed innovative irradiation methods to be developed which include the use of combinations of various wavelengths of ultraviolet light. By the selection and/or manufacture of light-emitting diodes having desired output wavelengths, UV illumination can be used for decontamination of specific materials and surfaces targeting microorganisms. Alternatively, or in addition, an active area of research is the use of UV irradiation to control viruses, parasites and fungal organisms.

The application of a magnetic field having a low field strength (10-500 Gauss) can be used to extend the lifetime of free radical entities. This low field strength has been described as a paradoxical low field effect, and arises as symmetry breaking favors singlet state degeneracy, and favors singlet to triplet interconverson with a range of vector states that are incompatible with recombination. The singlet/triplet interconversion is not favored by magnetic field strengths that exceed the hyperfine energy levels of the radicals. The energy level seen in the Zeeman effect relates to the hyperfine levels.

By extending free radical lifetimes through the imposition of a low strength magnetic field in this way, the effectiveness and/or efficiency of sanitizing and/or sterilizing objects and surfaces with UV light is enhanced. In one aspect of the present disclosure, the efficiency of UV-based sterilization processes can be increased by 30 to 60 percent, based on the reaction environment and quantum intersystem crossing among singlet and triplet states of the free radical entities.

The methods of the present disclosure can employ magnetic fields that may be either static or dynamic. An engineer with sufficient experience in electrochemical and electromagnetic processes would be well-able to construct an appropriate device and/or reaction chamber that could be used to simultaneously expose a selected substance or material with a desired wavelength of UV light while simultaneously subjecting the substance or material to an appropriate magnetic field.

The methods of the present disclosure may be used to sanitize and/or sterilize materials that are either static (still) or in motion. Where the material is static, the material can be placed in the reaction chamber manually, or by any conventional industrial conveyor, such as a conveyor belt or robotic handling system. Where the material is in motion, the material may be conveyed by any such suitable conveyor, but the material remains in motion.

Where the material to be sanitized and/or sterilized is liquid or semi-liquid, the material may flow through the reaction chamber. For example, the reaction chamber may include a flow-through cell where a flowing liquid or semi-liquid can be simultaneously exposed to UV light and a magnetic field.

The methods of the present disclosure may be used to sanitize and/or sterilize solid, liquid, or semi-solid materials. The materials being treated in this way may include foodstuffs, beverages, medicaments, medical devices, clothing, medical wastes, and any other material, without limitation.

The present methods are agnostic as to the source of UV radiation employed, provided that a magnetic field of the appropriate and specific magnetic strength is employed. In one aspect of the present disclosure, the strength of the magnetic field employed is between 0.01 and 5.0 Tesla. In another aspect of the method, the magnetic field is substantially uniform across the volume of the reaction chamber. The reaction chamber may be engineered with a suitable photonic UV source, and optionally incorporates one or more UV-reflective materials such as, for example, aluminum sheeting, panels, or foil.

The magnetic field applied to the reaction chamber can be generated by a magnetic field device. A magnetic field device typically includes a magnetic field generator, a magnetic field sensor, a data entry panel that permits a user to input time periods and electromagnetic field strengths for an electromagnetic field production regime, and a data processor including non-transitory computer readable memory, adapted to control said magnetic field generator to output a magnetic field in accordance with data received from said data entry panel.

The requisite magnetic field can be generated by any appropriate magnetic field source, such as for example ferrimagnets or Helmholtz coils. In one aspect, the magnetic field sources are arrayed such that the magnetic field can be contoured to fit the desired topography of the electrolytic apparatus. Alternatively, or in addition, one or more magnetic field sensors connected to the control station can be used to monitor the magnetic field in multiple axes. In one embodiment, the magnetic field sensors are Hall probes.

An engineer of ordinary background and education with regards to electromagnetic field generation devices can design and fabricate apparatus having electromagnetic coils that are capable of generating sufficiently uniform and/or contoured magnetic fields of the specific field strength, as described herein, and which would be suitable for the electrolytic methods described herein.

In the description and the claims, the term “substantially” means a deviation of up to 10% of the stated value, if physically possible, both downward and upward, otherwise only in the appropriate direction; in the case of degrees (angle and temperature), and for indications such as “parallel” or “normal,” this means ±10°. For terms such as “substantially constant” etc., what is meant is the technical possibility of deviation which the person skilled in the art proceeds from, and not the mathematical one. For example, a “substantially L-shaped cross-section” comprises two elongated surfaces, which merge at one end into the end of the other surface, and whose longitudinal extensions are arranged at an angle of 45° to 120° to each other.

All given quantities and percentages, in particular those relating to the limitation of the invention, insofar as they do not relate to specific examples, are understood to have a tolerance of ±10%; accordingly, for example: 11% means 9.9% to 12.1%. With terms such as “a solvent,” the word “a” is not to be considered to represent a singular numeral, but rather is to be considered an indefinite article or pronoun, unless the context indicates otherwise.

The term: “combination” and/or “combinations,” unless otherwise stated, mean all types of combinations, starting from two of the relevant components up to a plurality or all of such components; the term “containing” also means “comprising.”

Although the present methods and apparatus have been shown and described with reference to the foregoing operational principles and preferred embodiments, it will be apparent to those skilled in the art that various changes in form and detail may be made without departing from the spirit and scope of the invention. The present invention is intended to embrace all such alternatives, modifications and variances that fall within the scope of the appended claims.

Inventions embodied in various combinations and subcombinations of features, functions, elements, and/or properties may be claimed through presentation of new claims in related applications. Such new claims, whether they are directed to a different invention or directed to the same invention, whether different, broader, narrower or equal in scope to the original claims, are also regarded as included within the subject matter of the present disclosure.

What is claimed is:

1. A method of sanitization, comprising:
placing a material to be sanitized into a reaction chamber;
simultaneously exposing the material to be sanitized to an ultraviolet light selected to decrease a number of living or viable microorganisms in or on the material, and to a magnetic field having a field strength selected to prolong a lifetime of a free radical entity generated by the ultraviolet light exposure for a time sufficient to prolong the lifetime of the free radical entity;
removing the sanitized material from the reaction chamber.

2. The method of claim 1, wherein the magnetic field is created by at least one magnet positioned exterior to the electrochemical apparatus.

3. The method of claim 1, wherein exposing the electrochemical apparatus to the magnetic field includes exposing the electrochemical apparatus 10-500 gauss.

4. The method of claim 1, wherein a wavelength of the ultraviolet light includes one or more of UVA, UVB, and UVC.

5. The method of claim 1, wherein the material to be sanitized includes foodstuffs and/or beverages.

6. The method of claim 1, wherein the material to be sanitized includes medicaments and/or medical devices.

7. The method of claim 1, wherein the material to be sanitized includes clothing.

8. The method of claim 1, wherein the material to be sanitized includes medical waste.

9. The method of claim 1, wherein placing the material to be sanitized into the reaction chamber includes passing the material through the reaction chamber using a suitable conveyor.

10. The method of claim 1, wherein the material to be sanitized is a liquid or semi-liquid, and placing the material to be sanitized into the reaction chamber includes passing the liquid or semi-liquid through the reaction chamber within a flow-through cell.

11. The method of claim 1, wherein sanitizing the material includes sterilizing the material.

12. An apparatus for sanitizing a material, comprising:
a reaction chamber for receiving the material to be sanitized;
wherein the reaction chamber is configured to irradiate the material to be sanitized with an ultraviolet light selected to decrease a number of living or viable microorganisms in or on the material;
the reaction chamber is configured to expose the material to be sanitized to a magnetic field having a field strength selected to prolong a lifetime of a free radical entity generated by the ultraviolet light irradiation for a time sufficient to prolong the lifetime of the free radical entity; and
the irradiation of the material with the ultraviolet light and the exposure of the material to the magnetic field is substantially simultaneous.
